(19) United States
(12) Patent Application Publication
(10) Pub. No.: US 2014/0128412 A1
(43) Pub. Date: May 8, 2014

(54) COMBINATION OF AN ANTIPSYCHOTIC AND AN ANTI-INFLAMMATORY AGENT

(71) Applicant: Ru-Band Lu, Tainan (TW)

(72) Inventor: Ru-Band Lu, Tainan (TW)

(21) Appl. No.: 14/154,589

(22) Filed: Jan. 14, 2014

Related U.S. Application Data
(62) Division of application No. 13/039,774, filed on Mar. 3, 2011.

(51) Int. Cl. A61K 31/519; A61K 31/485

(52) U.S. Cl. CPC A61K 31/519; A61K 31/485
USPC 514/259.41

(57) ABSTRACT

The present invention relates to a pharmaceutical composition comprising an antipsychotic in combination with an anti-inflammatory agent, and to the use thereof in the treatment of psychotic disorder, and in particular, of schizophrenia.

### CROSS REFERENCE TO RELATED APPLICATION

This application claims priority to, and is a Divisional of, U.S. patent application Ser. No. 13/039,774, file on Mar. 3, 2011.

### FIELD OF THE INVENTION

The present invention relates to a pharmaceutical composition comprising an antipsychotic in combination with an anti-inflammatory agent.

### BACKGROUND OF THE INVENTION

Schizophrenia, a complex and severe brain disorder with a poorly defined etiology and pathophysiology, produces diverse disturbances in cognition, reality testing, mood, interpersonal relations, and social and work function. The disease is associated with a certain degree of central nervous degeneration. Some quantitative abnormalities in the brain structures of schizophrenic patients have been reported, e.g., enlarged ventricles, decreased cerebral and temporal lobe volumes, and thalamic anomalies. So far, the underlying pathophysiological cause and neuronal degeneration of schizophrenia is essentially unknown and should be studied. Increasing evidence suggests that abnormal cytokine levels in psychosis that activation of the cytokine-mediated inflammatory response system is important in the pathogenesis of neuronal degeneration and may be highly relevant to the symptoms and the progression of schizophrenia. For example, IL-1β, TNF-α etc., are proinflammatory cytokines that modulate synaptic plasticity and are involved in neurodegenerative processes in the brain. In addition, IL-1β and TNF-α sustain dopaminergic neuron degeneration in MPTP-induced parkinsonism animal models. Overexpressed IL-1β and TNF-β mRNA have been found in the peripheral blood mononuclear cells of schizophrenic patients. The cerebral spinal fluid (CSF) IL-1β level and serum IL-1β and IL-6 levels in schizophrenic patients were significantly higher than in healthy controls. Increased cytokine expression in plasma and the central nervous system indicates the activation of inflammation might contribute to the neuronal damage and degeneration in schizophrenia.

Moreover, accumulating evidence suggests that neurotrophic factors might be a candidate molecule involved in the pathophysiology of mental illness. For example, functional alterations in brain derived neurotrophic factor (BDNF) have recently been implicated in the pathophysiology of schizophrenia. BDNF, a basic dimeric protein, is a member of the nerve growth factor family, which is involved in neuronal survival, differentiation, synaptogenesis, and maintenance. It has to be maintained throughout life in order to preserve essential functions such as learning and memory. Studies have shown that BDNF is expressed in the postnatal brain, with the highest mRNA levels in the hippocampus and neocortex in rodents and humans. The mRNA levels of BDNF are relatively low during the infant and adolescent periods, reach a peak during young adulthood, and are maintained at a constant level throughout adulthood. Studies have shown that BDNF promoted the survival of a wide range of neuronal cells, such as the dopaminergic neurons of the substania nigra, cerebellar granule neurons, motoneurons, and retinal ganglion cells. The influence of BDNF on dopaminergic neurons may be associated with the pathogenesis of schizophrenia, which is suggested to be related to dopaminergic dysfunction. Because schizophrenia is a neuro-developmental disorder, the dysfunction of neurotrophins like BDNF might reflect the severity of neuronal dysfunction and degeneration of schizophrenia.

Accumulating evidence has associated BDNF and schizophrenia. A well-established prenatal stress animal model resembling schizophrenia features caused a reduction of BDNF expression in the prefrontal cortex and striatum and displayed persistent altered regulation of BDNF in the same brain structures in adult rats. Another neonatal ventral hippocampal lesion animal model of schizophrenia also caused a decrease in BDNF expression in the hippocampus and prefrontal cortex. Thus, it was suggested that corticostriatal and hippocampal BDNF dysregulation contributes to a permanent alteration of brain functions and leads to increased susceptibility to schizophrenia-like pathology. Taken together, the increase in cytokine expression and the decrease in BDNF expression in schizophrenic patients might reflect a certain degree of neuronal damage in schizophrenia. Anti-inflammatory or neurotrophic medicines may be beneficial when treating schizophrenia.

The current therapeutic approach to the treatment of schizophrenia focuses on the symptomatology. The most common therapy is a dopamine antagonist or combined dopamine and serotonin antagonists for their positive and negative symptoms. However, most typical and atypical antipsychotic drugs have a poor effect on the progress of the neurodegeneration that might relate to the negative symptoms of schizophrenia. Risperidone is an atypical antipsychotic that is both a dopamine and serotonin antagonist and has long been used to treat schizophrenia. In vitro studies have shown that atypical antipsychotic agents, including risperidone, significantly attenuated the production of interferon-γ induced proinflammatory cytokines, including IL-6, TNF-α etc. in microglial cells, and in mice following peripheral lipopolysaccharide administration.

Dextromethorphan (DM) has been used as an antitussive drug with few side effects for more than 50 years in clinics. DM is also an non-competitive N-methyl-D-aspartate (NMDA) receptor antagonist, which is neuro-protective for monoamine neurons. DM is neuroprotective for dopaminergic neurons and protects against endotoxicity. DM protects dopamine neurons against inflammation-mediated degeneration. The mechanism of the neuroprotective effect of DM is associated with the inhibition of microglia activation but not with its NMDA receptor antagonist property. It is well known that there is no correlation between the affinity of the NMDA receptor antagonist and DM analog 3-hydroxymorphinan and the potency of its neuroprotection for dopaminergic neurons. This suggests that DM's neuroprotection of dopaminergic neurons in inflammation-related neurodegenerative models is not mediated through the NMDA receptor but through its anti-inflammatory effects.

Recent studies have associated glial cells and their secretions, especially proinflammatory cytokines in plasma and CSF, with the development of mental illness. Previous studies have shown that plasma cytokines may access the brain through certain carriers or a damaged blood brain barrier. A systemically administered single dose of lipopolysaccharide that could not readily reach the brain induced early (1-9 h) serum and liver TNF-α expression, long-lasting (7-30 days) brain TNF-α expression, and, subsequently, caused neurodegeneration. Thus, peripheral inflammation may initiate or contribute to neuroinflammation and neurodegeneration in the central nervous system.

Reduced BDNF mRNA levels have also been reported in the prefrontal cortex and hippocampus of schizophrenic patients, as have reduced BDNF levels in the dorsolateral frontal cortex, anterior cingulated cortex, and hippocampus. In post-mortem brain tissue from schizophrenic patients, increased BDNF levels in cortical areas and decreased levels in the hippocampus had been found. In addition to these reports of reductions of BDNF expression in schizophrenic patients, there are also reports of unaltered and increased plasma BDNF expression.

### BRIEF DESCRIPTION OF THE DRAWINGS

FIG. 1 shows the plasma cytokine (TNF-α & IL-1β), CRP and BDNF level of normal control and schizophrenic patients (schizo) before medication treatment. *P<0.05, **p<0.01, ***p<0.001 vs normal control data.

FIG. 2 shows no significant difference in risperidone treatment dose (mg) between risperidone only (Risp, 2-6 mg/day) and risperidone combine dextromethorphan (DM, 60 mg/days) (Risp+DM) group.

FIG. 3 shows the plasma cytokine (TNF-α & IL-1β), CRP and BDNF level of schizophrenic patients after risperidone (Risp) or risperidone combine dextromethorphan (DM, 60 mg/days) (Risp+DM). Plasma sample were collected and analyzed at visit (v) and 0, 1, 2, 4, 6, 8 and 11 weeks. *P<0.05, vs visit (V) data at same group.

### SUMMARY OF THE INVENTION

The present invention relates to a pharmaceutical composition comprising an antipsychotic in combination with an anti-inflammatory agent, and to the use thereof in the treatment of psychotic disorder, and in particular, of schizophrenia.

### DETAILED DESCRIPTION OF THE INVENTION

The aims of the present invention is to investigate: (1) changes in the expression levels of proinflammatory factors (IL-1β, TNF-α, and CRP) and plasma BDNF in schizophrenic patients; (2) whether risperidone has an anti-inflammatory and neurotrophic effect, and (3) whether combining risperidone with DM produces a stronger anti-inflammatory and neurotrophic effect in schizophrenic patients. If neuroprotective therapy is efficacious for schizophrenia, that will provide a much better understanding of the biochemical pathology and treatment of schizophrenia.

The present invention find that schizophrenia is associated with upregulated plasma cytokine and CRP levels. The significant increase in the expression of inflammatory cytokines in schizophrenia implied the activation of the immunological response. The increased expression of peripheral cytokines in our schizophrenic patients indicated possible cytokine-induced neuronal damage.

The present invention also find downregulated plasma BDNF levels in schizophrenic patients, which indicates dysfunction of the neurotrophic system.

Because of the increase in inflammation and decrease in the neurotrophic response in the schizophrenic patients, anti-inflammatory and neurotrophic medications is beneficial in schizophrenia treatment. In the present invention, it is found that risperidone treatment reduced the elevated plasma IL-1β and CRP in schizophrenic patients. It reflects one of its therapeutic benefits. Moreover, the present invention find that risperidone treatment causes a significant increase in plasma TNF-α. It indicates that risperidone can also be toxic effect after long-term treatment. Thus, adding an anti-inflammatory agent like DM is beneficial adjuvant therapy for long-term risperidone treatment.

The present invention find that co-treatment of dextromethorphan with risperidone not only attenuates increases plasma IL-1β and CRP levels, but also attenuates risperidone-induced TNF-α production. Thus, a combination of dextromethorphan with risperidone can provide more benefit than risperidone alone for treating schizophrenia.

In the present invention, the plasma BDNF level does not change after 12 weeks of risperidone only or risperidone plus DM treatment. In the schizophrenic patients, baseline plasma BDNF levels were 73.4% lower than those of healthy controls before pharmacological treatment. It indicates serious neurotrophic system dysfunction and neuronal damage. Risperidone plus DM does not rescue the damaged neurotrophic function in these schizophrenic patients. Thus, risperidone plus DM treatment provides more neurotrophic and neuronal rescue effects for schizophrenic patients with normal baseline BDNF levels but not those with extremely low BDNF levels. Once the adult central nervous system is damaged, it is difficult to regenerate and needs long period of time to recover. A longer treatment time (months to years) is needed for schizophrenic patients with extremely low BDNF levels.

Accordingly, the present invention find increased inflammatory activity and decreased neurotrophic function in schizophrenic patients. In addition, the present invention shows that the plasma BDNF level is a biomarker for determining the severity of schizophrenia. Long-term use of the atypical antipsychotic risperidone attenuates inflammatory activity but also produces a certain degree of toxicity. Risperidone combined with DM provides more benefit and prevents the toxicity produced by long-term risperidone-only treatment. The present invention suggests that the unwanted toxic effects of different antipsychotic drugs in treating psychiatric disorders, especially schizophrenia attenuated by DM are via neural anti-inflammation process.

Thus, the present invention provides a pharmaceutical composition comprising an antipsychotic in combination with an anti-inflammatory agent, optionally in combination with a pharmaceutically acceptable carrier. Preferably, the antipsychotic is risperidone. Preferably, the anti-inflammatory agent is dextromethorphan. In a preferred embodiment, the pharmaceutical composition is a combination of risperidone and dextromethorphan.

The present invention also provides a method for treating a patient suffering from psychotic disorder, comprising administering to said patient an effective amount of a pharmaceutical composition comprising an antipsychotic in combination with an anti-inflammatory agent, optionally in combination with a pharmaceutically acceptable carrier. Preferably, the patient is suffering from schizophrenia. In a preferred embodiment, the patient is administered an effective amount of a combination of risperidone and dextromethorphan. The plasma IL-1β level of the patient administered the combination of risperidone and dextromethorphan is at least 3% lower than that of the patient administered risperidone alone. The plasma CRP level of the patient administered the combination of risperidone and dextromethorphan is at least 1% lower than that of the patient administered risperidone alone. The combination of risperidone and dextromethorphan further attenuates risperidone-induced TNF-α production of the patient compared with administering risperidone alone.

### EXAMPLES

The examples below are non-limiting and are merely representative of various aspects and features of the present invention.

### Example 1

### Methods

### Patient Selection

One hundred and thirty-seven Han Chinese schizophrenic patients and 67 Han Chinese healthy controls (age range: 20-59 years old) were recruited from the Department of Psychiatry at National Cheng Kung University Hospital and the National Defense Medical Center. All patients were screened for being used. The Chinese version of the SADS-L, which has good reliability and validity, was used to screen all participants for psychiatric conditions and whether they fit the DSM-IV diagnostic criteria for schizophrenia without any comorbidity. Exclusion conditions were: 1) pregnant or nursing women; 2) women of childbearing potential not using adequate contraception as per investigator judgment or not willing to use contraception for the duration of study; 3) patients who had used dextromethorphan, other selective cyclooxygenase 2 inhibitors, or other anti-inflammatory medication within 1 week before the first dose of double-blind medication; 4) a major mental illness other than schizophrenia, including alcoholism and illegal substance use disorder; 5) a clinically significant medical condition, e.g., cardiac, hepatic, or renal disease with current evidence of poor control; 6) patient who had undergone electroconvulsive therapy within 4 weeks before the first dose of double-blind medication; 7) SGOT, SGPT, BUN, and creatinine levels/an SGOT, SGPT, BUN, or creatinine level greater than 3-fold the upper limit of normal. All the healthy controls were free of major and minor mental illness. All research involving humans was reviewed and approved by the National Chung Kung University and National Defense Medical Center Institutional Review Boards before the study began.

### Study Design

This study was a double-blind, stratified, randomized, parallel-group, two-center clinical trial. The treatment was risperidone plus a double-blind add-on of randomly assigned DM or a placebo. Participants began the study immediately after they had been screened for eligibility (screening visit: V). The pre-treatment study phase consisted of a 7-day (V to 0 week) antipsychotic switch-over period for patients (patient hospitalized) who were taking oral antipsychotic medication other than risperidone alone at the screening, or who had been using depot medication for an entire cycle before the screening. The switchover period, which involved withdrawal of previous non-risperidone antipsychotics with the simultaneous titration of open-label risperidone, or a continuation of the existing risperidone dose if taken in combination with one or more other drugs at the screening, was followed by a one-week open-label risperidone-only treatment period, with the patient still hospitalized. At the end of the risperidone-only treatment week, patients were randomly assigned add-on DM (60 mg/day) or placebo treatment period. Patients already on risperidone alone at screen (visit), or not currently receiving any antipsychotic go directly into risperidone-only treatment period, then were randomly assigned DM or placebo add-on therapy. The add-on treatment period lasted for 11 weeks (77 days).

During the one-week open-label risperidone-only treatment period, patients were given between 1-6 mg/daily. Concomitant benzodiazepine (preferably up to 8 mg of lorazepam) was used for night-time sedation, agitation, or insomnia during the study.

### Main Outcome Measures

Ten milliliters of whole blood was drawn and collected from each participant's antecubital vein at the screening visit (V), 0, 1, 2, 4, 6, 8, and 11 weeks. Plasma was isolated from the whole blood after the 3000 g centrifugation at 4° C. for 15 min and immediately stored at −80° C. Plasma cytokine, CRP and BDNF concentrations were quantified using enzyme-linked immunosorbent assays (ELISA). A kit (Quantikine Human Cytokine kit; R&D Systems) and an ELISA reader (SpectraMax-M2; Molecular Devices) ELISA reader were used to analysis plasma IL-1β, TNF-α, CRP, and BDNF levels.

### Statistical Analyses

Data are means±SEM. One-way analysis of variance (ANOVA) followed by the Newman-Keuls test was used for the statistical evaluations. Significance was set at p<0.05.

### Results

### Patients

One hundred and thirty-seven male and female patients with acute schizophrenia were enrolled. Forty-two (30.7%) patients dropped out during the 12-week trial, while 95 patients completed the 12 weeks of treatment. The data for plasma IL-1β, TNF-α, CRP, and BDNF level measurements were from the 95 schizophrenic patients and 67 healthy controls.

### Efficacy

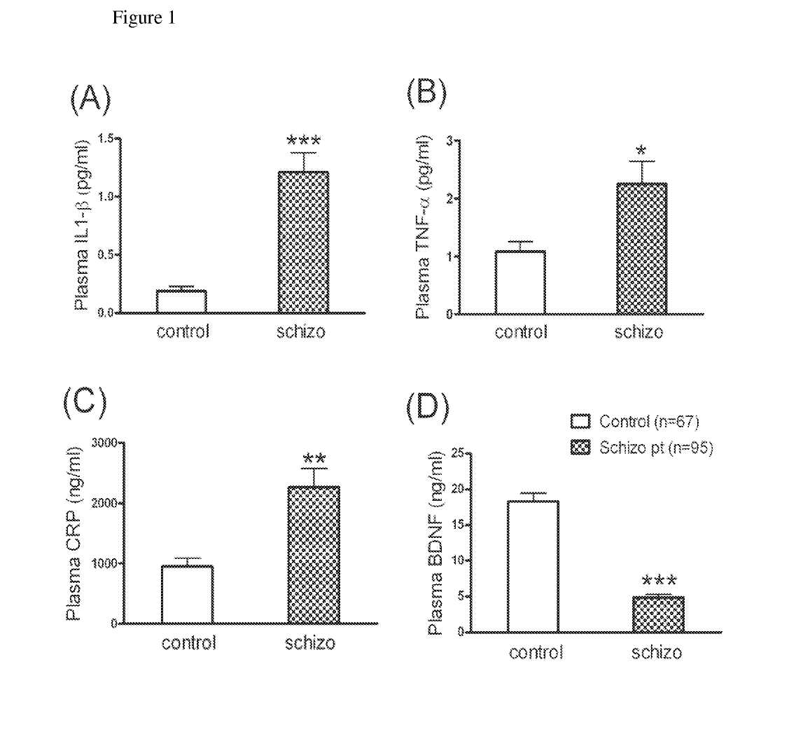

Schizophrenic patients had significantly higher levels of plasma IL-1β (1.21±0.17 vs. 0.19±0.04 pg/ml; p<0.001), TNF-α (2.25±0.39 vs. 1.09±0.16 pg/ml; p<0.05), and CRP (2265.0±314.8 vs. 951.6±132.0 pg/ml; p<0.01), and significantly lower levels of plasma BDNF (4.88±0.47 vs. 18.32±1.13 ng/ml; p<0.001) than did healthy controls (FIG. 1).

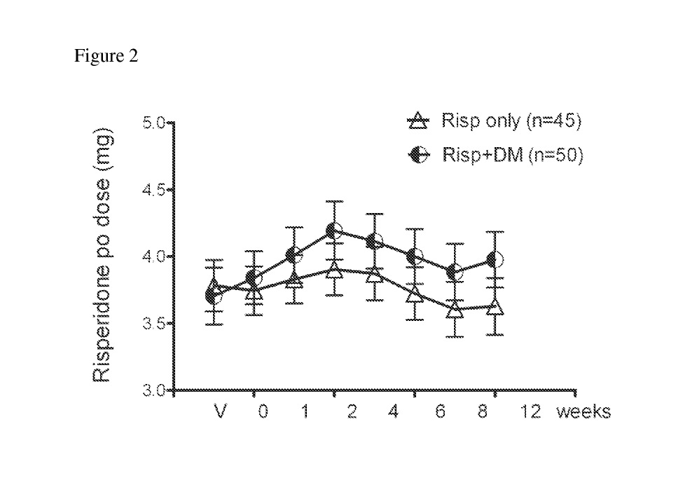

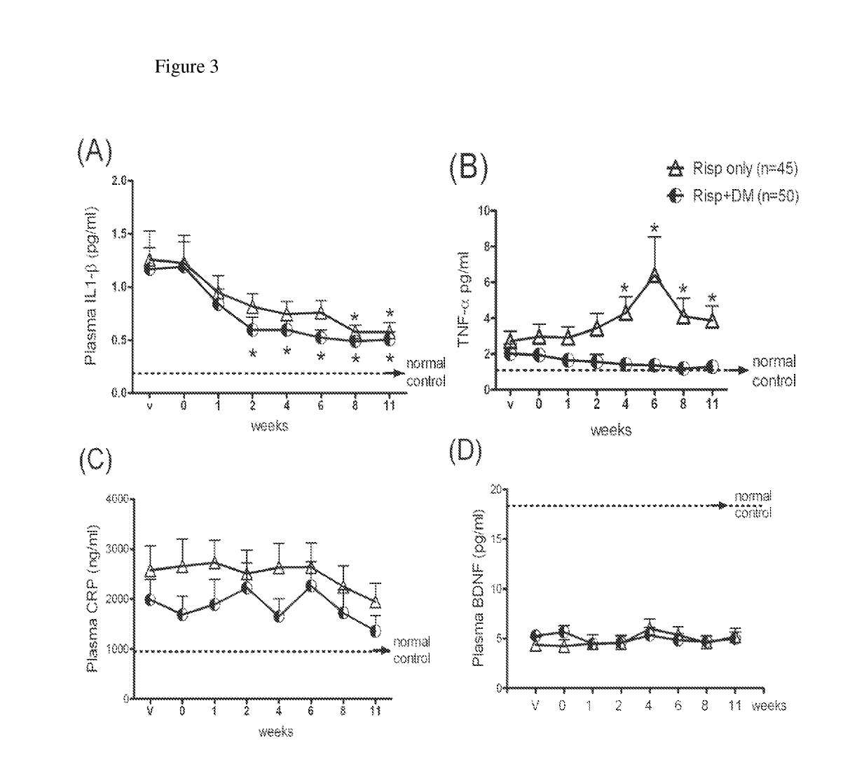

An average of 3-4 mg of risperidone was used to treat schizophrenia. There was no significant difference in the risperidone dose between the risperidone only (Risp only) and risperidone plus DM (Risp+DM) treatment group from the initial screening visit (V) to 11 weeks (FIG. 2). In the inflammatory factors analysis, there was a significant decrease in plasma IL-1β levels in the Risp only and Risp+DM treatment groups (FIG. 3A) after 11 weeks of treatment. Moreover, IL-1β level fell after only 2 weeks in the Risp+DM group compared with after 8 weeks in the Risp only group. Plasma TNF-α levels in the Risp+DM group fell continuously during the study; however, in the Risp only group, plasma TNF-α levels rose significantly after 4 weeks, peaked at 6 weeks, and were significantly higher at the end of the study (FIG. 3B). Plasma CRP levels tended to decrease but were not significantly different in the Risp only group (2576.0±484.0 to 1946.0±369.6) or in the Risp+DM group (1986.0±410.0 to 1359.0±317.5) (FIG. 3C). After 11 weeks of treatment, plasma BDNF levels did not change in the either the Risp only group or the Risp+DM group (FIG. 3D).

One skilled in the art readily appreciates that the present invention is well adapted to carry out the objects and obtain the ends and advantages mentioned, as well as those inherent therein. The composition, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for treating a patient suffering from psychotic disorder comprising administering to said patient an effective amount of a pharmaceutical composition comprising an antipsychotic in combination with an anti-inflammatory agent, optionally in combination with a pharmaceutically acceptable carrier.

2. The method of claim 1, wherein the antipsychotic is risperidone.

3. The method of claim 1, wherein the anti-inflammatory agent is dextromethorphan.

4. The method of claim 1, wherein the pharmaceutical composition is a combination of risperidone and dextromethorphan.

5. The method of claim 1, wherein the patient is suffering from schizophrenia.

6. The method of claim 1, wherein the plasma IL-1β level of the patient is at least 3% lower than that of the patient administered risperidone alone.

7. The method of claim 1, wherein the plasma CRP level of the patient is at least 1% lower than that of the patient administered risperidone alone.

8. The method of claim 1, which attenuates risperidone-induced TNF-α production of the patient compared with administering risperidone alone.

9. The method of claim 8, which reduces TNF-α level of the patient to normal level of TNF-α.
